(19) United States
(12) Patent Application Publication
(10) Pub. No.: US 2003/0055013 A1
(43) Pub. Date: Mar. 20, 2003

(54) HCV combination therapy

(75) Inventor: Clifford A. Brass, Scotch Plains, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: SCHERING CORPORATION

(21) Appl. No.: 10/247,396

(22) Filed: Sep. 19, 2002

Related U.S. Application Data
(60) Provisional application No. 60/323,619, filed on Sep. 20, 2001.

(51) Int. Cl. A61K 38/21; A61K 31/7056; A61K 31/375; A61K 31/426

(52) U.S. Cl. 514/43; 424/85.7; 424/764; 424/702; 514/562; 514/474; 514/458; 514/369; 514/762; 514/725

(57) ABSTRACT

Methods of treating patients having susceptible viral infections, especially chronic hepatitis C infection by administering to said patient a therapeutically effective amount of a combination therapy of interferon-alfa and ribavirin for a time sufficient to lower HCV-RNA in association with a therapeutically effective amount of an antioxidant therapy comprising s-adenosyl methionine, preferably S-adenosyl L-methionine, for a time sufficient to ameliorate ribavirin-related hemolysis are disclosed.

### BACKGROUND OF THE INVENTION

The present invention relates to methods of treating patients having susceptible viral infections, especially chronic hepatitis C infections by administering to said patient a therapeutically effective amount of a combination therapy of interferon-alfa and ribavirin for a time sufficient to lower HCV-RNA in association with a therapeutically effective amount of an antioxidant therapy comprising S-adenosyl methionine for a time sufficient to ameliorate ribavirin-related hemolysis.

A chronic hepatitis C viral infection is a particularly insidious and slow-progressing viral disease having a significant impact on the quality of life. It can eventually result in cirrhosis of the liver, decompensated liver disease and/or hepatocelluar carcinoma.

Combination treatment with interferon alfa-2b and ribavirin for chronic hepatitis C in patients. is disclosed by Reichard et al.(The Lancet 1998; 351;83-87. T. Poynard et al.(The Lancet, 1998, Vol. 352, October 31, p 1426-1432) disclose that treating chronic hepatitis C patients who had not been treated with interferon or ribavirin with 3 MIU of interferon alfa-2b TIW plus 1000-1200 mg of ribavirin per day for 48 weeks resulted in a sustained virological response at 24 weeks after treatment in 43% of the patients. See also J. G. McHutchinson et al. (N. Engl. J. Med., 1998, 339:1485-1492), G. L. Davis et al. (N. Engl. J. Med., 1998, 339:1493-1499) disclose that treating chronic hepatitis C patients who relapsed after treatment with interferon with 3 MIU of interferon alfa-2b TIW plus 100-1200 mg of ribavirin per day for 48 weeks results in higher rates of sustained virologic response than treatment with interferon alone.

However this combination therapy is not always effective due to side effects associated ribavirin such as ribavirin-related hemolysis as measured by reduced hemoglobin concentrations. Both McHutchinson, et al and Poynard, et al report that the majority of patients who completed the combination therapy had reached their lowest hemoglobin concentration by the fourth week of combination therapy at which time the hemoglobin concentrations either stabilized or increased. Ribavirin does reduction to 600 mg/day was reported by McHutchinson, et al for patients with hemoglobin concentrations below 10 g per deciliter and treatment with ribavirin was discontinued in patients with hemoglobin concentration below 8.5 g per deciliter. Ribavirin dose reduction or does cessation was necessary in certain patients and resulted in subsequent increases in hemoglobin, but lower sustained virologic reponses.

Brass, C, et al. disclosed in Gastroenterology Suppl., Vol. 116, No. 4, Al 192, Abstract L0056, April 1999 a pilot study of patients having chronic hepatitis C who received the antioxidants vitamin C and vitamin E in combination with the FDA-approved combination therapy of ribavirin and interferon alf-2b, but Brass, et al do not disclose the present invention.

There is a need to, provide an improved combination therapy for treating susceptible viral infections, especially chronic hepatitis C patients, to ameloriate the ribavirin-related hemolysis throughout the duration of the combination therapy especially in the first 4 to 12 weeks, of therapy so as to produce a sustained virological response in more patients than previously possible.

### SUMMARY OF THE INVENTION

The present invention provides methods for treating susceptible viral infections, especially hepatitis C viral infections. which comprises administering to said patient a therapeutically effective amount of ribavirin for a time sufficient to lower viral serum levels in association with a therapeutically effective amount of an antioxidant therapy comprising S-adenosyl methionine for a time sufficient to ameliorate ribavirin-related hemolysis.

The present invention provides a method of treating a patient having chronic HCV infection which comprises administering to said patient a therapeutically effective amount of a combination therapy of interferon-alfa and ribavirin for a time sufficient to lower HCV-RNA serum levels in association with a therapeutically effective amount of an antioxidant therapy comprising S-adenosyl methionine for a time sufficient to ameliorate ribavirin-related hemolysis.

The present invention provides a method of treating a patient having a chronic HCV infection which comprises administering to said patient a therapeutically effective amount of a combination therapy of pegylated interferon alfa and ribavirin sufficient to lower detectable HCV-RNA in association with a therapeutically effective amount of an antioxidant therapy comprising S-adenosyl methionine sufficient to ameliorate ribavirin-related hemolysis.

In a preferred embodiment, the therapeutically effective amount of the combination therapy of interferon alfa and ribavirin is administered for a period of about 24 weeks for patients with HCV genotype 2 or 3, or for a period of about 48 weeks for patients with HCV genotype 1.

The preferred the antioxidant therapy comprises Vitamin C, Vitamin E, and S-adenosyl methionine, or a pharmaceutically acceptable ester or salt thereof.

The present invention also provides a method of treating a patient having a chronic HCV infection which comprises administering to said patient a therapeutically effective amount of a combination therapy of pegylated interferon alfa and ribavirin for a time period sufficient to lower detectable HCV-RNA in association with a therapeutically effective amount of an antioxidant therapy sufficient to ameliorate ribavirin-related hemolysis.

The time period is at least about 24 weeks, and more preferably is at least about 48 weeks or 72 weeks The preferred pegylated interferon-alfa is pegylated interferon-alfa-2a, pegylated interferon-alfa-2b; pegylated consensus interferon or pegylated purified interferon-alfa product. The preferred pegylated interferon-alfa is pegylated interferon-alfa-2a or pegylated interferon-alfa-2b; the use of pegylated interferon-alfa-2b is more preferred.

In a preferred embodiment, the present invention relates to a method of treating a patient having a chronic HCV infection which comprises administering to said patient a combination therapy of about 0.5 to about 3.0 μg/kg/week of pegylated interferon alfa-2b and about 600 to about 2000 mg/day of ribavirin for a time period sufficient to lower detectable HCV-RNA in association with a therapeutically effective amount of an antioxidant therapy sufficient to ameliorate ribavirin-related hemolysis of at least about 24 weeks. In a more preferred embodiment, about 800 to about 1400 mg/day of ribavirin are administered depending on the weight of said patient.

In another preferred embodiment, the present invention relates to a method of treating a patient having a chronic HCV infection which comprises administering to said patient a combination therapy of about 0.5 to about 3.0 μg/kg/week of pegylated interferon alfa-2b and about 600 to about 2000 mg/day of ribavirin for a time period sufficient to lower detectable HCV-RNA in association with a therapeutically effective amount of an antioxidant therapy sufficient to ameliorate ribavirin-related hemolysis. In a more preferred embodiment, about 800 to about 1400 mg/day of ribavirin are administered depending on the weight of said patient.

In a preferred embodiment, the present invention relates to a method of treating a patient having a chronic HCV infection which comprises administering to said patient a combination therapy of about 0.5 to about 3.0 μg/kg/week of pegylated interferon alfa-2b and about 600 to about 2000 mg/day of ribavirin in association with a therapeutically effective amount of an antioxidant therapy sufficient to ameliorate ribavirin-related hemolysis. In a more preferred embodiment, about 800 to about 1400 mg/day of ribavirin are administered depending on the weight of said patient.

In a preferred embodiment, the present invention relates to a method of treating a patient having a chronic HCV infection which comprises administering to said patient a combination therapy of about 0.5 to about 3.0 μg/kg/week of pegylated interferon alfa-2b and about 13 mg/kg/day of ribavirin in association with a therapeutically effective amount of an antioxidant therapy sufficient to ameliorate ribavirin-related hemolysis.

The preferred antioxidant therapy comprises S-adenosyl methionine, Vitamin and Vitamin C. The preferred Vitamin E derivatives include, but are not limited to, d-alpha-tocopheerol and esters thereof, for example, the water soluble d-alpha-tocopheryl polyethylene glycol esters such as the water dispersible Vitamin E d-alpha-tocopheryl polyethylene glycol 1000 succinate (“Vitamin E-TPGS”) as well as use of compositions of Vitamin E-TPGS and at least one fatty acid ester of glycerine having an overall melting point of 40° C. (both of which are disclosed in U.S. Pat. No. 5,234,695 and available from Eastman Kodak Co., Rochester, N.Y.).

### DETAILED DESCRIPTION

The present invention provides methods for treating susceptible viral infections, especially hepatitis C viral infections.

The term “susceptible viral infections” as used herein means viral infections caused by a wide range of RNA and DNA viruses, including, but not limited to, the families of viruses such as flaviruses-including the genus flavirus, pestivirus of which Kunjin virus is a member, and hepavirus of which hepatitis C virus is a member, and arbovirus of which the West Nile virus is a member-orthomyxoviruses, paramyxoviruses, arenaviruses, bunyaviruses, herpes viruses, adenoviruses, poxviruses, and retroviruses.

Typical suitable “susceptible viral infections” include influenza A and B viral infections; parainfluenza viral infections, respiratory syncytial virus(“RSV”) infections such as RSV bronchiolitis and RSV pneumonia especially such RSV infections in children and infants as well as RSV pneumonia in patients with preexisting cardiopulmonary disease, measles viral infections, Lassa fever viral infections, Korean Haemorrhagic fever infections, hepatitis B viral (HBV) infections, Crimean Congo-Haemorrhagic and HCV infections and HIV-1 infections, encephalitis infections such as caused by West Nile virus or Kunjin virus or the St. Louis encephalitis infections as well as viral infections found in immunocompromised patients. Other susceptible viral infections are disclosed in U.S. Pat. No. 4,211,771 at column 2, line 21 to column 3 line 37; doses and dose regimens and formulations are disclosed at column 3, line 4 to column 9, line 5;

see also Canadian Patent No. 1,261, 265. Sidwell, R. W., et al. Pharmacol. Ther., 1979, Vol. 6 pp 123-146 discloses that the in vivo antiviral experiments conducted with ribavirin generally confirm one broad-spectrum antiviral activity seen in vitro and states that the efficacy of ribavirin is quite dependent upon the site of infection; the manner of treatment; the age of the animal and the virus dosage utilized. Tables 4 and 5 on page 127 list the RNA and DNA virus infections significantly inhibited in vivo by ribavirin.

In the original clinical studies of the combination therapy of interferon alfa-2b and ribavirin Rebetron, the most notable laboratory adverse event was anemia. A reduction in hemoglobin of greater than 4 g/dL occurred in 14% of patients in the 6 month duration study and in 21% of the patients in the 12 month clinical trial. Ribavirin monotherapy also resulted in a reduction in hemoglobin of greater than 4 g/dL in 15.7% of patients with chronic hepatitis C in prior studies. The proportion of patients experiencing hemoglobin reductions of greater than 4 g/dL is, therefore, similar for ribavirin mono or the combination therapy. Among the patients who started therapy with baseline hemoglobin within the normal range a reduction to below 10 g/dL was uncommon. The majority of patients had reached their lowest hemoglobin value by the first month of therapy at which time the hemoglobin stabilized or increased. Ribavirin dose reduction or dose cessation was necessary in certain patients and resulted in subsequent increases in hemoglobin.

In a preferred embodiment, the present invention provides an improved method of treating patients having hepatitis C infection by administrating a therapeutically effective amount of an antioxidant therapy to ameloriate the ribavirin-related hemolysis so as to allow such patients to continue using the therapeutically effective amount of combination therapy until a sustained virologic response is achieved. The combination therapy includes, but is not limited to (a) interferon alfa-2b and ribavirin and (b) pegylated interferon alfa 2b and ribavirin such as approved by the FDA as well as other interferon alfa and ribavirincombination therapies under clinical development, e.g., pegylated interferon alfa 2a and ribavirin or those described hereinafter.

The combination therapy is continued for a time period of at least about 24 weeks, more preferably is at least about 48 weeks or 72 weeks, and most preferably is at least about 48 weeks.

The patients treated in accordance with the preferred embodiments should have no detectable HCV-RNA at the end of such time periods, and also should have no detectable HCV-RNA for at least 24 weeks after the end of such time period.

The present method of treating patients having chronic hepatitis C infections allows delivery of therapeutically effective amount of the combination of ribavirin and of interferon-alfa, especially in the first 4 to 12 weeks of combination therapy, sufficient to substantially lower detectable HCV-RNA serum levels, preferably by at least two powers of ten, i.e., at least 102 lower than the initial HCV-RNA serum level, and more preferably eradicate detectable HCV-RNA serum levels.

The term “antioxidant” as used herein means a substance that delays or inhibits hemolysis related to or promoted by administration of ribavirin.

The term “a therapeutically effective amount of an antioxidant” as used herein means an amount of antioxidant in the range of about one to about two hundred and fifty times, preferably about one to about two hundred times, more preferably ten to about one hundred times, most preferably ten to about fifty times the recommended daily dietary allowance (“RDA”) of antioxidants (or the recommended daily amount if no RDA is reported for an antioxidant) useful in the methods of the present invention.

The antioxidant therapy useful in the methods of the present invention will normally be administered for at least 24 weeks, and preferably as long as the ribavirin, administered as part of the combination therapy, and/or ribavirin monotherapy is delivered to the patient having susceptible viral infections, especially hepatitis C infections. The continued administration of the antioxidant therapy beyond 24 weeks of treatment in accordance with the present invention while preferred will be at the discretion of the attending clinician and the patient. In a preferred embodiment of the present invention, the antioxidant therapy is initiated at least two weeks, more preferably four to six weeks, and most preferably four weeks before administration of the ribavirin monotherapy or combination therapy is initiated.

The term “in association with” as used herein in reference to administration of ribavirin monotherapy or the combination therapy of interferon-alfa and ribavirin with an antioxidant means that the antioxidant is administered prior to, concurrently with, or after administration of the combination therapy. The antioxidant may be administered orally, parenterally (e.g. IM, IP, SC or IV) or topically, e.g. by suppository. Oral or parenteral (e.g. subcutaneous) administration is preferred. Oral administration is more preferred. Typically, the antioxidant is administered in single or divided doses concurrently with the ribavirin which may be administered in single or divided doses BID.

Typically, suitable antioxidants include Vitamin A, Vitamin E, Vitamin C, silybum marianum, co-enzyme-Q10, BHA, BHT, 2-oxo-4-thiazolidinecarboxylic acid, N-acetylcysteine, S-adenosyl methionine, selenium, panavir, lycopene, or pharmaceutically acceptable salts and esters thereof.

A preferred embodiment of the antioxidant therapy of the present invention comprises S-adenosyl methionine, more preferably S-adenosyl L-methionine. Another preferred embodiment of the antioxidant therapy of the present invention comprises S-adenosyl methionine, and at least one of Vitamin A, Vitamin E, Vitamin C, silybum marianum, co-enzyme-Q10, BHA, BHT, 2-oxo-4-thiazolidinecarboxylic acid, N-acetylcysteine, selenium, panavir, lycopene, or pharmaceutically acceptable salts and esters thereof. Another preferred embodiment of the antioxidant therapy of the present invention comprises S-adenosyl methionine, Vitamin E, and Vitamin C, or pharmaceutically acceptable salts and esters thereof.

Vitamin A is described in Merck Index 11th Edition #9918 (and 4919) at pages 1576-77. The RDA is in the range of about 250 International Units (“IU”)/day up to about 2500 IU/day; preferably about 2500 IU/day for newborns and infants.

The term “Vitamin E” as used herein includes all forms of tocopherol including, but not limited to, the naturally occurring and synthetic homologues of the four types of tocopherols (alpha-, beta-, gamma- and delta-tocopherol) and four types of tocotrienols, including esters thereof, e.g., alpha tocopheryl acetate or alpha tocopheryl succinate as well as the dextrorotatoary (“d”), levorotatoary (“I”) optical isomers or mixtures of the d and I isomers. The d optical isomers are more active than I isomers and use of the optical isomers of vitamin E is preferred. The effective amount of Vitamin E useful in the present invention is in the range of one to two hundred and fifty (250) times, preferably one to one hundred times the recommended daily dietary allowance (“RDDA”) of 3 to 10 alpha tocopherol equivalents per day; i.e., 3-10 mg of d-alpha tocopherol per day. The activity of 1 mg of d-alpha tocopherol is equal to 1 alpha tocopherol equivalents. Since 1 mg of d-alpha tocopherol is equivalent to 1.49 International units (IU) of Vitamin E, about 4.5 to about 15 IU of Vitamin E is the RDDA. The activity of the following Vitamin E derivatives has been measured: one IU of Vitamin E is equivalent to 1 mg of dl-alpha tocopheryl acetate, 1 mg of d-alpha tocopheryl acetate has the potency of 1.36 IU of Vitamin E, 1 mg of d-alpha tocopherol has the potency of 1.49 IU of Vitamin E; and 1 mg of d-alpha tocopheryl succinate has the potency of 1.21 IU of Vitamin E;. See Goodman & Gilman's The Pharmacological Basis of Therapeutics, Ninth Edition, 1996, McGraw-Hill, pages 1549 and 1585-1590.

Suitable Vitamin E derivatves include d-alpha-tocopherol (available from Roche, Nutley, N.J. with a recommended daily allowance, of 10-30 mg per day equal to 200 IU of Vitamin E per day for adults), Vitamin E esters such as Vitamin E acetate and succinate (alpha-tocopheryl acetate and alpha-tocopheryl succinate) as well as water soluble Vitamin E derivatives. Use of water soluble Vitamin E derivatives is preferred.

Water soluble Vitamin E derivatives include pharmaceutically acceptable Vitamin E salts such as Vitamin E phosphate, as well as water soluble tocopheryl polyethylene glycol esters such as those disclosed in U.S. Pat. Nos. 2,680,749, 3,914,430 and 5,234,695; water soluble tocopheryl polyethylene glycol esters are preferred.

Use of d-alpha-tocopheryl polyethylene glycol esters such as the water dispersible Vitamin E d-alpha-tocopheryl polyethylene glycol 1000 succinate(“Vitamin E-TPGS”) as well as use of compositions of Vitamin E-TPGS and at least one fatty acid ester of glycerine having an overall melting point of 40° C. (both of which are disclosed in U.S. Pat. No. 5,234,695 and available from Eastman Kodak Co., Rochester, N.Y.) are more preferred.

The effective amounts of Vitamin E derivatives, e.g., water soluble Vitamin E derivatives such as Vitamin E-TPGS is in the range of about 200 IU to 20000 IU of Vitamin E/day, preferably about 1000 IU to about 5000 IU of Vitamin E/day, more preferably about 1200 IU to about 2200 IU of Vitamin E/day, in single or divided doses. Dimitrov, N V, et al., Am J Clin Nutr(USA) September 1996, Vol. 64, (3) pages 329-335 reports that administration of 400 IU (269 mg), 800 IU (537 mg) and 1200 IU (807 mg) of Vitamin E-TPGS to healthy volunteers as a single oral dose resulted slight elevation of plasma alpha tocopherol concentrations. Sokool, R J, et al. Gastroenterology, (USA), June 1993, Vol. 104(6) pages 1727-1735 report that a dose of 20-251U/kg/day of Vitamin E-TPGS appears to be safe and effective for treating chronic childhood cholestasis. The RDA of Vitamin E-TPGS for pediatrics is in the range of 15-251U/kg/day.

Vitamin C (L-ascorbic acid and D-ascorbic acid) is readily available as an over the counter product. See also Merck Index, 11th Edition #855 pp. 130-131. The RDA for Vitamin C is in the range of about 100 to about 200 mg/day, but the use of doses of up to 25 g/day or even 40 g/day have been reported.

Silybum marianum (silymarin), the active ingredient in milk thistle, is available from Magdaus A G, Germany under the LEGALON trade name. The RDA for silymarin is 140 mg/day, capsule, PO.

Coenzyme -Q10 is a vitamin-like substance made by the human body and is also found in organ meats. The RDA for coenzyme -Q10 is about 300 to 400 mg/day.

BHA (butylated hydroxyanisole) (Merck Index, 11th Edition, No. 1547) and BHT (butylated hydroxytolune (Merck Index, 11th Edition, No. 1548 p. 238) are available from Aldrich Chemical Company Inc., Milwaukee, Wis. 53233.

2-Oxo-4-thiazolidinecarboxylate (also 2-oxo-thiazolidine-4-carboxylate) and known as procysteine) used in the present invention may be in the D or L form or mixtures thereof, including the DL racemate. The use of the L form is preferred. The synthesis of the L- and D-stereoisomeric forms is disclosed in the Merck Index Eleventh Edition No. 7084 at page 1193 and by Kaneko, et al. Bull, Chem Soc. (Japan) by modified by Shah, et al in Cancer Research, Vol. 39, pp 3942-3947 (1979). Pharmaceutical compositions of L-2-oxo-thiazolidine-4-carboxylate are preferably in the form of its neutral alkali metal salt, e.g., sodium or potassium or alkaline earth salt e.g. magnesium in a concentration of 0.25 to 2.5 g/dl and are disclosed in U.S. Pat. No. 4,647,571.

The dose of procysteine is in the range of about 200 to about 1000 mg/day preferably about 200 to about 800 mg/day or about 300 to about 600 mg/day.

Acetyl cysteine (N-Acetyl-L-cysteine, “NAC”) is found in the body. See also Merck Index, 11th Edition, No. 82 p. 14 The RDA for NAC is in the range of about 300 mg/day to 600 mg/day.

S-adenosyl methionine used in the present invention may be in the D or L form or mixtures thereof, including the DL racemate. The use of the L-form is preferred and is available from Sigma, P.O. Box 14508, St. Louis, Mo. 63178 USA. Pharmaceutical acceptable salts include the chloride and the p-toluene sulfonate salts.

The dose of S-adenosyl-L-methionine is at least 400 mg/day, preferably in the range of about 400 to 3000 mg/day, more preferably about 1000 to about 2400 mg/day or about 1000 to about 1800 mg/day or about 1200 to about 1800 mg/day in single or divided doses. Use of divided doses of 300, 400, 500 mg or 600 mg two or three times a day is preferred. The use of 400 or 600 mg three times a day is more preferred.

Selenium is available from Aldrich Chemical Company Inc., Milwaukee, Wis. 53233. The RDA for selenium is 200 to 600 micrograms/day.

Panavia (4,4′-isopropylidenedithiobis-2,6-di-t-butylphenol) is available from VyrexCorporation, LaJolla, Calif. 92037 (USA). The RDA for Panavia 200 mg to 800 mg/day PO for HIV patients; higher doses stablized or slightly stabilized CD4 levele in Phase I/II clinical trials. See PharmaProjects, section J5A

Lycopene is found in tomatoes; see NY TIMES, Apr. 13, 1999, Science Times Section page F 12. The RDA for lycopene is in the range of 5 to 15 mg/day.

Pharmaceutical compositions of the antioxidants suitable for oral, parenteral and topical administration and useful in the present invention may contain the excipient, and other ingredient found in the over the counter preparations of the antioxidants. Compositions of Vitamin E-TPGS and at least one fatty acid ester of glycerine having an overall melting point of 40° C. disclosed in U.S. Pat. No. 5,234,695 may also be used. See also the compositions of Trolox and Trolox C disclosed in J Pharm Pharmacol(GB), Feb 1995, Vol 47(2), pages 138-142.

The in vitro inhibitory concentrations of ribavirin are disclosed in Goodman & Gilman's “The Pharmacological Basis of Therapeutics”, Ninth Edition, (1996) McGraw Hill, NY, at pages 1214-1215. The Virazole product information discloses a dose of 20 mg/mL of Virazole aerosol for 18 hours exposure in the 1999 Physicians Desk Reference at pages 1382-1384.

Ribavirin dosage and dosage regimens are also disclosed by Sidwell, R. W., et al. Pharmacol. Ther 1979 Vol 6. pp123-146 in section 2.2 pp 126-130. Fernandes, H., et al., Eur. J. Epidemiol., 1986, Vol 2(1) pp1-14 at pages 4-9 disclose dosage and dosage regimens for oral, parenteral and aerosol administration of ribavirin in various preclinical and clinical studies.

The term “patients having hepatitis C infections” as used herein means any patient-including a pediatric patient-having hepatitis C and includes treatment-naive patients having hepatitis C infections and treatment-experienced patients having hepatitis C infections as well as those pediatric, treatment-naive and treatment-experienced patients having chronic hepatitis C infections.

These patients having chronic hepatitis C include those who are infected with multiple HCV genotypes including type 1 as well as those infected with, inter alia, HCV genotype 2 and/or 3.

The term “pediatric patient” as used herein means a patient below the age of 17, and normally includes those from birth to 16 years of age.

The term “treatment-naive patients having hepatitis C infections” as used herein means patients with hepatitis C who have never been treated with ribavirin or any interferon, including but not limited to interferon-alfa, or pegylated interferon alfa.

The term” treatment-experienced patients having hepatitis C infections” as used herein means patients with hepatitis C who have been treated with ribavirin or any interferon, including but not limited to interferon-alfa, or pegylated interferon alfa, including relapsers and non-responder.

The term “patients having chronic hepatitis C infections” as used herein means any patient having chronic hepatitis C and includes “treatment-naive patients and treatment-experienced patients having chronic hepatitis C infections, including but not limited to relapsers and non-responders.

The term “relapsers” as used herein means treatment-experienced patients with hepatitis C who have relapsed after initial response to previous treatment with interferon alone, or in combination with ribavirin.

The term “non-responders” as used herein means treatment-experienced patients with hepatitis C who have not responded to prior treatment with any interferon alone, or in combination with ribavirin.

The term “interferon-alfa” as used herein means the family of highly homologous species-specific proteins that inhibit viral replication and cellular proliferation and modulate immune response. Typical suitable interferon-alfas include, but are not limited to, recombinant interferon alfa-2b such as Intron-A interferon available from Schering Corporation, Kenilworth, N.J., recombinant interferon alfa-2a such as Roferon interferon available from Hoffmann-La Roche, Nutley, N.J., recombinant interferon alpha-2c such as Berofor alpha 2 interferon available from Boehringer Ingelheim Pharmaceutical, Inc., Ridgefield, Conn., interferon alpha-n1, a purified blend of natural alfa interferons such as Sumiferon available from Sumitomo, Japan or as Wellferon interferon alpha-n1 (INS) available from the Glaxo-Welicome Ltd., London, Great Britain, or a consensus alpha interferon such as those described in U.S. Pat. Nos. 4,897,471 and 4,695,623 (especially Examples 7, 8 or 9 thereof) and the specific product available from Amgen, Inc., Newbury Park, Calif., or interferon alfa-n3 a mixture of natural alfa interferons made by Interferon Sciences and available from the Purdue Frederick Co., Norwalk, Conn., under the Alferon Tradename or recombinant interferon alpha available from Frauenhoffer Institute, Germany or that is available from Green Cross, South Korea. The use of interferon alfa-2a or alpha 2b is preferred. Since interferon alpha 2b, among all interferons, has the broadest approval throughout the world for treating chronic hepatitis C infection, it is most preferred. The manufacture of interferon alpha 2b is described in U.S. Pat. No. 4,530,901.

The term “pegylated interferon-alfa” as used herein means polyethylene glycol modified conjugates of interferon-alfa, preferably pegylated interferon alfa-2a, pegylated interferon alfa-2b, or a pegylated consensus interferon, more preferably pegylated interferon alfa-2a and pegylated interferon alfa-2b. The preferred polyethylene-glycol-interferon alfa-2b conjugate is PEG₁₂₀₀₀-interferon alfa 2b. The phrases “12,000 molecular weight polyethylene glycol conjugated interferon alpha” and “PEG₁₂₀₀₀-IFN alfa” as used herein mean conjugates such as are prepared according to the methods of International Application No. WO 95/13090 and containing urethane linkages between the interferon alfa-2a or -2b amino groups and polyethylene glycol having an average molecular weight of 12000.

The preferred PEG₁₂₀₀₀-interferon alfa-2b is prepared by attaching a PEG polymer to the epsilon amino group of a lysine residue in the IFN alfa-2b molecule. A single PEG₁₂₀₀₀ molecule is conjugated to free amino groups on an IFN alfa-2b molecule via a urethane linkage. This conjugate is characterized by the molecular weight of PEG₁₂₀₀₀ attached. The PEG₁₂₀₀₀-IFN alfa-2b conjugate is formulated as a lyophilized powder for injection. The objective of conjugation of IFN alfa with PEG is to improve the delivery of the protein by significantly prolonging its plasma half-life, and thereby provide protracted activity of IFN alfa.

Other interferon alfa conjugates can be prepared by coupling an interferon alfa to a water-soluble polymer. A non-limiting list of such polymers include other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alfa-polymer conjugates are described in U.S. Pat. No. 4,766,106, U.S. Pat. No. 4,917,888, European Patent Application No. 0 236 987, European Patent Application Nos. 0510 356, 0 593 868 and 0 809 996 (pegylated interferon alfa-2a) and International Publication No. WO 95/13090.

Pharmaceutical compositions of pegylated interferon alfa-suitable for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g. NaCl), preservatives (e.g. thimerosol, cresol or benylalcohol), and surfactants(e.g. tween or polysorabates) in sterile water for injection. The pegylated interferon alfa-may be stored as lyophilized powders under a refrigeration at 2°-8° C. The reconstituted aqueous solutions are stable when stored between 2° and 8° C. and used within 24 hours of reconstitution. See for example U.S. Pat. Nos. 4,492,537; 5,762,923 and 5,766,582. The reconstituted aqueous solutions may also be stored in prefilled, multi-dose syringes such as those useful for delivery of drugs such as insulin. Typical suitable syringes include systems comprising a prefilled vial attached to a pen-type syringe such as the NOVOLET Novo Pen available from Novo Nordisk, as well as prefilled, pen-type syringes which allow easy self-injection by the user. Other syringe systems include a pen-type syringe comprising a glass cartridge containing a diluent and lyophilized pegylated interferon alfa powder in a separate compartment.

The following preferred embodiments for administering therapeutically effective amounts of the combination therapy of interferon alfa and ribavirin are presented

The interferon-alpha administered as part of the combination therapy is preferably selected from interferon alpha-2a, interferon alpha-2b, a consensus interferon, a purified interferon alpha product or a pegylated interferon-alpha. More preferably, the interferon-alpha is selected from interferon alpha-2a, interferon alpha-2b, or a purified interferon alpha product and the amount of interferon-alpha administered is from 2 to 10 million IU per week on a weekly, three times a week (“TIW”), every other day (“QOD”) or daily basis. In a preferred embodiment, the interferon-alpha administered is interferon-alpha-2b and the amount of interferon-alpha is administered 3 million IU TIW.

Alternatively, the interferon-alpha administered as part of the combination therapy is consensus interferon and the amount of interferon-alpha administered is from 1 to 20 micrograms per week on a weekly, BIW,TIW, QOD or daily basis. In another embodiment, the interferon-alpha administered is a pegylated interferon alpha-2b and the amount of interferon-alpha administered is from 0.5 to 2.0 micrograms per week on a weekly, BIW, TIW, QOD or daily basis. Alternatively, the interferon-alpha administered is a pegylated interferon alpha-2a and the amount of interferon-alpha administered is from 20 to 250 micrograms/kilogram per week on a weekly, TIW, QOD or daily basis.

When the pegylated interferon-alfa administered as part of the combination therapy is a pegylated interferon alfa-2b, the therapeutically effective amount of pegylated interferon alfa-2b administered during the treatment in accordance with the present invention, is in the range of about 0.1 to 9.0 micrograms per kilogram of pegylated interferon alfa-2b administered per week, in single or divided doses, preferably once a week (QW) or twice a week(BIW), preferably in the range of about 0.1 to about 9.0 micrograms per kilogram of pegylated interferon alfa-2b administered once a week (QW) or in the range of about 0.05 to about 4.5 micrograms per kilogram of pegylated interferon alfa-2b administered twice a week(BIW), or is in the range of about 0.5 to about 3.0 micrograms per kilogram of pegylated interferon alfa-2b administered per week, preferably in the range of about 0.5 to about 3.0 micrograms per kilogram, or about 1.5 to about 3.0 micrograms per kilogram of pegylated interferon alfa-2b administered once a week (QW) or in the range of about 0.25 to about 1.5 micrograms per kilogram of pegylated interferon alfa-2b administered twice a week, or is in the range of about 0.75 to about 1.5 micrograms per kilogram of pegylated interferon alfa-2b administered per week, most preferably is in the range of about 0.75 to about 1.5 micrograms per kilogram of pegylated interferon alfa-2b administered once a week or about 0.375 to about 0.75 micrograms per kilogram of pegylated interferon alfa-2b administered twice a week.

When the pegylated interferon-alfa administered to pediatric patients as part of the combination therapy is a pegylated interferon alfa-2b, the therapeutically effective amount of pegylated interferon alfa-2b administered during the treatment in accordance with the present invention, including in first and second treatment time periods is in the range of about 0.1 to 9.0 micrograms per kilogram of pegylated interferon alfa-2b administered per week, in single or divided doses, preferably once a week (QW) or twice a week(BIW), more preferably about 0.1 to about 9.0 micrograms per kilogram of pegylated interferon alfa-2b administered once a week (QW), or about 0.05 to about 4.5 micrograms per kilogram of pegylated interferon alfa-2b administered per week, in single or divided doses, preferably once a week (QW) or twice a week(BIW), more preferably about 0.05 to about 4.5 micrograms per kilogram of pegylated interferon alfa-2b administered once a week, or preferably about 0.75 to about 3.0 micrograms per kilogram of pegylated interferon alfa-2b administered in single or divided doses, preferably once a week (QW) or twice a week (BIW), more preferably about 0.75 to about 3.0 micrograms per kilogram, or about1.5 to about 3.0 micrograms per kilogram of pegylated interferon alfa-2b administered once a week or about 0.375 to about 1.5 micrograms per kilogram of pegylated interferon alfa-2b administered twice a week, and most preferably about 2.25 to about 2.6 micrograms per kilogram of pegylated interferon alfa-2b administered once a week or about 1.1 to about 1.3 micrograms per kilogram of pegylated interferon alfa-2b administered twice a week(BIW).

When the pegylated interferon-alfa administered as part of the combination therapy is a pegylated interferon alfa-2a, the therapeutically effective amount of pegylated interferon alfa-2a administered during the treatment in accordance with the present invention, is in the range of about 50 micrograms to about 500 micrograms once a week(QW), preferably about 180 micrograms to about 250 micrograms QW or the effective amount is in the range of about 50 micrograms to about 250 micrograms twice a week, preferably about 90 micrograms to about 125 micrograms twice a week.

When the pegylated interferon-alfa administered to a pediatric patient as part of the combination therapy is a pegylated interferon alfa-2a, the therapeutically effective amount of pegylated interferon alfa-2a administered during the treatment in accordance with the present invention, including in first treatment time period is in the range of about 50 micrograms to about 500 micrograms once a week(QW), preferably about 300 micrograms to about 375 micrograms QW or the therapeutically effective amount of pegylated interferon alfa-2a administered to a pediatric patient is in the range of about 50 micrograms to about 250 micrograms twice a week, preferably about 150 micrograms to about 190 micrograms once a week

Ribavirin is administered as part of the combination therapy to the patient in association with pegylated interferon-alfa, that is, before, after or concurrently with the administration of the pegylated interferon alfa. The pegylated interferon-alfa dose is preferably administered during the same period of time that the patient receives doses of ribavirin.

The term “therapeutically weigh-effective amount of ribavirin” means an amount that is sufficient to produce a sustained virologic response for at least about twelve weeks post treatment, preferably for at least about twenty-four weeks post treatment, most preferably forty eight weeks post treatment.

The therapeutically weigh-effective amount of ribavirin administered concurrently with the pegylated interferon-alfa is from preferably about 400 to about 2000 mg per day, or about 600 to about 1200 mg/day or about 800 to about 1200 mg day and most preferably about 1000 to about 1200 mg/kg a day. The pegylated interferon-alfa dose is also preferably administered to the pediatric patient during the same period of time that such patient receives doses of ribavirin. The therapeutically weigh-effective amount of ribavirin administered to the pediatric patient concurrently with the pegylated interferon-alfa is from about 8 to about 15 mg per kilogram per day, preferably about 8, 12 or 15 mg per kilogram per day, in divided doses.

In a preferred embodiment of the present invention, therapeutically weight-effective amount of ribavirin is in the range of at least about 13 mg to about 14.5 mg/kg of ribavirin per day, preferably at least about 13 mg/kg of ribavirin per day. In another preferred embodiment, the preferred therapeutically weight-effective amount of ribavirin is 800 mg/day for people having a weight of less than 65 kg, 1000 mg/day for people having a weight in the range of 65 kg to 85 kg, and 1200 mg/day for people having a weight greater than 85 kg.

A person suffering from chronic hepatitis C infection may exhibit one or more of the following signs or symptoms:

(a) elevated ALT,

(b) positive test for anti-HCV antibodies,

(c) presence of HCV as demonstrated by a positive test for HCV-RNA,

(d) clinical stigmata of chronic liver disease,

(e) hepatocelluar damage.

To practice the invention, the therapeutically effective amount of the combination therapy of interferon-alfa and ribavirin is administered to the patient exhibiting one of more of the above signs or symptoms in amounts sufficient to eliminate or at least alleviate one or more of the signs or symptoms. The therapeutically effective amount of the antioxidant therapy is administered in association with the therapeutically effective amount of the combination therapy o to ameliorate the ribavirin-related hemolysis.

Ribavirin is administered to the patient in association with the interferon-alfa, that is, the interferon-alfa dose is administered during the same period of time that the patient receives doses of the ribavirin derivative of the present invention. Most interferon-alfa formulations are not effective when administered orally, so the preferred method of administering the interferon-alfa is parenterally, preferably by subcutaneous, IV, or IM, injection. Ribavirin may be administered orally in capsule or tablet form in association with the parenteral administration of interferon-alfa. Of course, other types of administration of both medicaments, as they become available are contemplated, such as by nasal spray, transdermally, by suppository, by sustained release dosage form, etc. Any form of administration will work so long as the proper dosages are delivered without destroying the active ingredient.

Pharmaceutical composition of interferon-alfa, suitable for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human serum albumin), toxicity agents (e.g. NaCl), preservatives (e.g. thimerosol, cresol or benylalcohol), and surfactants(e.g. tween or polysorabates) in sterile water for injection. The interferon alfa-may be stored as lyophilized powders under a refrigeration at 2°-8° C. The reconstituted aqueous solutions are stable when stored between 20 and 8° C. and used within 24 hours of reconstitution. See for example U.S. Pat. Nos. 4,492,537; 5,762,923 and 5,766,582.

The term “no detectable HCV-RNA” in the context of the present invention means that there is less than 100 copies of HCV-RNA per ml of serum of the patient as measured by quantitative, multi-cycle reverse transcriptase PCR methodology. HCV-RNA is preferably measured in the present invention by the methodology described below. This methodology is referred to herein as HCV-RNA/qPCR.

The term “substantially lower detectable HCV-RNA serum levels” in the context of the present invention means that the HCV-RNA serum level is lower by at least a power of ten, preferably lower by two powers of ten and most preferably lower by at least three powers of ten, compared to the initial HCV-RNA serum level.

RNA is extracted from patient serum using a guaninidium thiocyanate-phenol-chloroform mister followed by ethanol-ammonium acetate precipitation. The precipitated RNA is centrifuged and the resulting pellet is dried in a Centrivap console (Labconco, Kansas City, Mo.). The dry pellet is then resuspended in 30 microliters of an Rnasin (Promega Corp., Madison, Wis.), dithiothritol, and diethylpyrocarbonate-treated water mixture. Samples are kept at or below −20° C. (preferably below −70° C.) until RNA reverse transcription (RT) and PCR.

In order to convert the entire RNA sequence into cDNA in the RT reaction, random hexadeoxyribonucleotides (Pharmacia Biotech, Piscataway, N.J.) are used as primers for the first strand cDNA synthesis. Two aliquots of 3 microliters of resuspended sample is added to 3 microliters of 100 ng/μl random primers and denaturated at 70° C., then reverse transcribed at 40° C. for one hour using M-MLV reverse transcriptase (USB, Cleveland, Ohio) in standard buffer containing 5 mM MgCl₂. The final RT reaction volume is 26 μl. The PCR is started immediately following the reverse transcription.

A modified version of the PCR method is performed using heat-stable Taq polymerase to amplify the cDNA. Seventy-five microliters of PCR mix is added to the entire RT reaction volume (26 μl) to a final MgCl₂ concentration of 1.5 mM in a total volume of 101 μl. Each 101 μl sample is then split into 50.5 μl, and a layer of mineral oil is placed on top to prevent evaporation.

The PCR cycle consists of annealing for 90 sec., extension for 90 sec., and denaturation for 90 sec., at 55° C., 74° C. and 94° C., respectively. Thermocycling samples is submitted to a final 74° C. extension for 10 minutes. Four different cycle sets are used. By loading the sample in duplicate, and splitting these samples evenly after RT, there are four tubes from one sample. Each of the four tubes is given a different cycle number, enhancing sensitivity and accuracy in the quantitation process. The thermocycling efficiency will be assessed by satisfactory amplification of known copy number RNA standards included in each set of 60 tubes. Two primer sets are used for the amplification, both from the 5′ untranslated region of the HCV genome. Both of these primer sets are highly conserved and detect all known subtypes of HCV. Primer set 1: upstream 5′-GTG GTC TGC GGA ACC GGT GAG T-3′, downstream 5′-TGC ACG GTC TAC GAG ACC TC-3′ which produced a 190 bp product. Primer set 2: upstream 5′-CTG TGA GGA ACT ACT GTC TTC-3′, downstream 5′-CCC TAT CAG GCA GTA CCA CAA-3′ which produced a 256 bp product.

The amplified cDNA is then electrophorised in 3% agarose gel and transferred to nylon membrane. The target DNA is detected by Southern blotting and immunostaining using a nonradioactive digoxigenin-labeled DNA probe. These procedures are performed using automated instruments for PCR thermocycling, agarose gel electrophoresis, vacuum-transfer Southern blot, hybridization, and immunostaining. Each membrane contains known copy number serially diluted standards which are used to construct standard curves for quantitative measurement of the specimen bands. Originally standard curves are made from carefully diluted HCV-RNA from transcribed clones. Radioactive incorporation studies, gel electrophoresis, and OD 260 are performed on the transcripts to determine that they are of the expected length. After the production of the RNA transcripts quantitated clone standards “pooled” standards are generated which better represent the heterogeneous nature of HCV, one would encounter in natural infection. These pools are made by combining large amounts of serum or plasma from known infected individuals. The serum/plasma pools are calibrated with PCR, against the clone transcripts and then diluted in the known PCR-negative fluids. Finally, the higher copy number samples of the pools are checked against the cDNA Quantiplex nucleic acid detection system from Chiron Inc. (Emeryville, Calif.). These “double quantitated” pools are aliquoted and saved at −70° C. Dilutions of 5,000,000, 1,000,000, 500,000, 100,000, 10,000, and 1000 copies/ml are used in each experiment.

Each Southern blot membrane is scanned into a computer using an automated scanner/densitometer, at intervals during development to determine when the standard curve is most linear. The resultant electronic images are then measured for band area and mean band density. All of the reading are standardized to integrated band density and compared to the standard curve to obtain a numerical value of viral copy number for each band.

The term “sustained virologic response” as used in the context of the present invention means that there is no detectable HCV-RNA in the patients treated in accordance with the present invention for at least 24 weeks after the end of the combined therapy treatment. Preferably, the period of sustained virologic response will be at least one year—or longer—after the end of treatment.

During treatment and post-treatment follow-up, biochemical (ALT), virological (HCV-RNA), hematology, including at least the following hemoglobin (HgB), hematocrit (HCT). RBC, WBC with differential and platelet counts) levels and histological (liver biopsy) examinations would be used to assess the nature and duration of response to study treatment. The primary efficacy variable of the clinical study described herein below will be a reduction in the drop of hemoglobin from baseline as compared to control at week 12 of antiviral therapy. The preferred primary efficacy variable will be the overall response defined as loss of serum HCV-RNA/qPCR (<100 copies/mL) as measured at 24 weeks following the end of therapy. In addition, the drop in the hemoglobin levels compared to baseline values will also be measured. In addition, a decrease in hepatic inflammation, an improvement in post-treatment liver biopsy as measured by the Knodell Histology Activity index (HAI) and normalization of ALT will also be examined as a secondary efficacy endpoints. The safety of the study treatments will be assessed by monitoring selected laboratory parameters and by also recording and evaluating the occurrence of any adverse events.

Efficacy

The primary efficacy objective will be to determine if the use of the antioxidant therapy can reduce the hemolysis that occurs with the use of ribavirin in the combination therapy(Arm B) as determined by a reduction in the drop of hemoglobin from baseline as compared to control (Arm A)at week 12 of antiviral combination therapy The following secondary Endpoints will also be examined using logistic regression:

The secondary Endpoints:

Number of patients requiring dose reduction or discontinuation of ribavirin;

Antioxidant measurements;

HCV RNA/qPCR measurement at week 24 of the antiviral combination therapy; and

Virology: Entry Status and Change from Entry

Serum HCV-RNA/qPCR testing and HCV genotype testing will be performed by a central laboratory. A positive HCV-RNA assay result will be required at Baseline; only patients positive for HCV-RNA will be eligible to participate. Repeat assays should be scheduled at Weeks 4, 12, 24, and if the patient is in the 48 week treatment groups at weeks 36 and 48. All patients should have repeat assays scheduled for Follow-up Weeks 12 and 24.

Response will be assessed as defined below:

| | |
|---|---|
| Virologic Responder: | A patient will be classified as a responder |
| | at a given time point if HCV-RNA/qPCR is |
| | negative (<100 copies per mL) at that time |
| | point. |
| Sustained Virologic | A patient will be classified as a sustained |
| Responder: | responder if the patient is a responder at |
| | 24 weeks of follow-up. |
| | Note that patients who do not meet these |
| | criteria, including patients who discontinued |
| | before the required HCV-RNA/qPCR |
| | evaluations are obtained, will be classified |
| | as non-responders. |
| Overall Responder: | Based on both serum HCV-RNA/qPCR and |
| | change in liver histology as evaluated by the |
| | Knodell HAI Inflammation Score. A patient |
| | will be classified as an overall responder to |
| | treatment if at 24 weeks of follow-up, he/she |
| | is a sustained virologic responder and has |
| | normal ALT. |

Liver Histology

Liver biopsy will be taken within the thirty six months preceding patient enrollment and at Follow-up Week 24 for all patients. Evaluation of the biopsies will be performed by a single pathologist using the Knodell Histology Activity Score.

The central pathologist will be blinded with respect to patient identification, treatment group, and the time the biopsy will be obtained relative to treatment (Pre- or Post-treatment). Repeat liver biopsy will not be required for patients with previously documented cirrhosis.

The patient's weight and their baseline disease characteristics (HCV genotype, hemoglobin levels and initial viral load) for all patients will be measured before the start of the study. HCV genotypes should be done on the patient serum samples subjected to HCV-RNA/qPCR testing.

This enhancement of efficacy included all aspects of the disease will result in:

Sustained eradication of detectable HCV-RNA;

Improvement in hepatic inflammation;

Lower Hemoglobin Drops;

Normalization of ALT;

Improvement in HQL.

### Clinical Study Design

This is a treatment protocol designed to examine the efficacy the antioxidant therapy of Vitamin E, Vitamin C, S-Adenosyl-L-Methionine in the amelioration of hemolysis associated with PEG-Intron+Ribavirin) for treatment of patients with chronic hepatitis C.

The following double blind treatment arms will be randomly assigned to patients at selected centers under this protocol:

| | |
|---|---|
| Arm A: | PEG-interferon alfa-2b 1.5 ug/kg once a week (QW) |
| | Ribavirin 800-1400 mg/day PO, |
| Arm B: | PEG-interferon alfa-2b 1.5 ug/kg QW |
| | Ribavirin 800-1400 mg/day + |
| | Antioxidant therapy: |
| | Vitamin E 800 International Units (IU)/day PO |
| | Vitamin C 1,000 IU/day PO |
| | S-adenosyl L-methionine 400 mg, three times a day (TID) |

Arm B patients will begin the antioxidant therapy 4 weeks prior to initiation of the 48 week course of the antiviral combination therapy. The antioxidant therapy will be continued through week 24 of antiviral therapy. Continued use of antioxidant therapy will then be at the discretion of the investigator and the patient.

Patients will be treated for a period of 48 weeks. Combination Therapy will be discontinued for patients not achieving a complete virologic response at 24 weeks.

Patients must weigh >85 kg to receive 1200 mg/day of ribavirin. Patients who weigh >105 kg will normally receive 1400 mg/day of ribavirin

### Objectives

The primary objective of this treatment protocol is to confirm that antioxidant therapy of Vitamin E, Vitamin C, S-Adenosyl-L-Methionine can reduce the hemolysis that occurs with the use of ribavirin, as determined by a reduction in the drop of hemoglobin from baseline compared to control at week 12 of the combination therapy.

The secondary objectives are to determine:

if there is a decrease in the number of patients needing ribavirin dose reductions;

if there is a decrease in patient discontinuations from therapy and an improved quality of life;

and to measure the antioxidant measurements -Erythrocyte: GSH, MDA, 4-HNE in select subgroup (10 patients in each arm);

the correlation of HgB drop with cholesterol level, platelet count; and

the HCV RNA/qPCR serum measurements at week 24 of the antiviral combination therapy.

### Study Synopsis

The protocol is a randomized study of PEG-Intron 1.5 mcg/kg/once a week (“QW”) and ribavirin 800-1400 mg/day(“QD”)+/−antioxidant therapy (Vitamin E 8001U/day, Vitamin C 1,000 IU/day, S-adenosyl-L-Methionine 400 mg/three times a day (“TID”). Pre-menopausal women that have documented the ability to menstruate within the past 2 years will be stratified prior to randomization. Therapy for all patients will continue for 48 weeks. Important: Arm B patients will begin anti-oxidant therapy 4 weeks prior to starting PEG-Intron+ribavirin. Therapy will be discontinued for patients not achieving a complete virologic response at 24 weeks. Safety and tolerance will be evaluated at 0, 2,4,8,12,16,20, and 24 weeks and then every 8 weeks until the end of therapy. Evaluations will be done at weeks 12 and 24 post therapy. Additional laboratory measurement of antioxidants in a select sub-group (10 patients in each group) will be done at baseline wk 2, 4, 8, and week 12 of antiviral therapy. These measurements include erythrocyte-reduced glutathione-GSH, malonyldialdehyde-MDA, and 4-hydroxynonenal-4-HNE. Correlation of HgB drop with cholesterol level, platelet count will also be assessed. Antioxidant therapy will begin 4 weeks prior to initiating anti-viral combination therapy.

### Study Population

Adult male and female patients with compensated, chronic hepatitis C who have not received previous treatment with interferon, PEG-interferon, ribavirin, or combination interferon+ribavirin will be selected for this study. Patients meeting the following Inclusion Criteria will be enrolled.

Inclusion Criteria:

Patients must be willing to give written informed consent and be able to adhere to dose and schedule.

Adult patients 18-70 years of age of either gender and any race.

Serum positive for HCV-RNA by PCR (qPCR) assay, or bDNA

Liver biopsy within 36 months prior to entry to this protocol with a pathology report confirming that the histological diagnosis is consistent with chronic hepatitis. Biopsy must score or be re-read to score fibrosis stage. Repeat biopsy not required for documented cirrhosis.

Compensated liver disease with the following minimum hematologic, biochemical, and serologic criteria at the Entry Visit (WNL=within normal limits):

Hemoglobin values of ≧12 gm/dL for females and ≧13 gm/dL for males.

WBC >3,000/mm3

Neutrophil count ≧1,500/mm3

Platelets ≧70,000/mm3

Direct bilirubin, within normal limits (WNL)

Indirect bilirubin, WNL (unless non-hepatitis related factors such as Gilbert's disease explain an indirect bilirubin rise. In such cases indirect bilirubin must be ≦3.0 mg/dL [≦51.3 μmol/L]).

Albumin, WNL

Serum creatinine, within 20% of ULN.

Fasting glucose should be 70-115 mg/dL. Results between 115-140 mg/dL requires repeat fasting glucose to be <140 mg/dL and HbA1c ≦8.5%. Hemoglobin A1c must be ≦8.5% for diabetic subjects (whether on medication and/or diet controlled).

Thyroid Stimulating Hormone (TSH), WNL (Subjects requiring medication to maintain TSH levels in the normal range are eligible if all other inclusion/exclusion criteria are met.).

HIV Negative

Serum hepatitis B surface antigen (HBsAg) negative

Patient weight <125 kg

Non-smokers within 3 weeks of study entry

Alpha fetoprotein value ≦100 ng/mL obtained within one year prior to entry. Patients with an alpha fetoprotein value >20 ng/mL but <100 ng/mL may be enrolled after a normal ultrasound within the previous 6 months. Eligible patients with AFP values ≧100 ng/mL and <300 must have a spiral CT scan or MRI which is negative for evidence of hepatocellular carcinoma. Eligible patients with AFP ≧300 ng/mL who do not have radiographic evidence of hepatocellular carcinoma may be enrolled with the approval of the Principal Investigator.

Reconfirmation and documentation that sexually active female subjects of childbearing potential are practicing adequate contraception (intrauterine device, oral contraceptives, progesterone implanted rods [Norplant], medroxyprogesterone acetate [Depo-Provera], surgical sterilization, barrier method [diaphragm+spermicide], or monogamous relationship with a male partner who has had a vasectomy or is using a condom+spermicide) during the treatment period and for six months following the last dose of study medication. A pregnancy test obtained at entry prior to the initiation of treatment must be negative. Female subjects must not be breast feeding.

Reconfirmation that sexually active male subjects are practicing acceptable methods of contraception (vasectomy, use of a condom+spermicide, monogamous relationship with a female partner who practices an acceptable method of contraception) during the treatment period and for six months following the last dose of study medication.

Exclusion Criteria

The patient will be excluded from entry if any of the following criteria apply:

Women who are pregnant or nursing.

Prior treatment with any interferon, PEG-interferon, ribavirin or combination interferon+ribavirin.

Prior treatment for hepatitis with any other antiviral or immunomodulatory drug within the previous 2 years.

Suspected hypersensitivity to interferon, PEG-interferon or ribavirin.

Participation in any other clinical trial within 30 days of entry to this protocol. Treatment with any investigational drug within 30 days of entry to this protocol.

Any other cause for the liver disease other than chronic hepatitis C including but not limited to:

Co-infection with HBV

Hemochromatosis (iron deposition >2+ in liver parenchyma)

Alpha-1 antitrypsin deficiency

Wilson's disease

Autoimmune hepatitis

Alcoholic liver disease

Obesity-induced liver disease

Drug-related liver disease

Hemophilia or any other condition that would prevent the subject from having a liver biopsy, including anticoagulant therapy.

Hemoglobinopathies (e.g., Thalassemia).

Evidence of advanced liver disease such as history or presence of ascites, bleeding varices, spontaneous encephalopathy.

Subjects with organ transplants other than cornea and hair transplant.

Any known preexisting medical condition that could interfere with the subject's participation in and completion of the protocol such as:

Preexisting psychiatric condition, especially severe depression, or a history of severe psychiatric disorder, such as major psychoses, suicidal ideation and/or suicidal attempt are excluded. Severe depression would include the following: (a) subjects who have been hospitalized for depression, (b) subjects who have received electroconvulsive therapy for depression, or (c) subjects whose depression has resulted in a prolonged absence of work and/or significant disruption of daily functions. Subjects with a history of mild depression may be considered for entry into the protocol provided that a pretreatment assessment of the subject's mental status supports that the subject is clinically stable. The Investigator will formulate a management plan for each of the subjects which will become a part of the subject's medical record. The management plan maybe developed in conjunction with a health care professional trained in psychology. For these subjects, the Investigator will review the subject's mental status at every visit.

CNS trauma or active seizure disorders requiring medication.

Significant cardiovascular dysfunction within the past 12 months (e.g., angina, congestive heart failure, recent myocardial infarction, severe uncontrolled hypertension or significant arrhythmia). Subjects with ECG showing clinically significant abnormalities.

Poorly controlled diabetes mellitus.

Chronic pulmonary disease (e.g., chronic obstructive pulmonary disease), documented pulmonary hypertension.

Immunologically mediated disease (e.g., inflammatory bowel disease [Crohn's disease, ulcerative colitis], rheumatoid arthritis, idiopathic thrombocytopenia purpura, systemic lupus erythematosus, autoimmune hemolytic anemia, scleroderma, severe psoriasis, clinical cryoglobulinemia with vasculitis).

Any medical condition requiring, or likely to require during the course of the study, chronic systemic administration of steroids.

Clinical gout.

Substance abuse, such as alcohol (>26 gm/day), IV drugs and inhaled drugs. If the subject has a history of substance abuse, to be considered for inclusion into the protocol, the subject must have abstained from using the abused substance for at least 6 months. Stable subjects enrolled in a methadone maintenance program for at least one year may be enrolled if they are otherwise eligible and are monitored throughout the study for illicit drug use.

Subjects not willing to be counseled/abstain from the consumption of alcohol.

Subjects with clinically significant retinal abnormalities.

Any other condition which in the opinion of the Investigator would make the subject unsuitable for enrollment, or could interfere with the subject participating in and completing the protocol.

Vitamin supplement use within 2 weeks of entering study

Oral or inhalation use of tobacco within 3 weeks of study entry

Use of EtOH in excess of 26 gms/day (2 drinks/day)

Entry Visit

Subjects will undergo an entry evaluation to confirm their eligibility to participate in the study. All results of the entry evaluations, except HCV-RNA/qPCR are required prior to the subject starting study medication. The initiation of therapy must start within 4 weeks of the Entry Visit.

The medical history, hepatitis disease history, physical exam/GI/Liver exam, ECG (when indicated), and ocular exam (if needed) will be recorded at the Entry Visit for this protocol.

Study Medication

Dosage of Study Medications

Patients will be treated with the doses of combination therapy ofPEG-Intron 1.5 mcg/kg QW and ribavirin 800,1,000, 1200 or 1400 mg QD +/−Vitamin E 800 IU/day, Vitamin C 1,000 IU/day, S-Adenosyl-L-Methionine 400 mg TID as determined by randomization.

Supplies of Ribavirin

Ribavirin will be supplied until it is available commercially from Schering Corporation, Kenilworth, N.J. as a separate agent or in combination with PEG-intron

Administration of Ribavirin

TABLE A
Ribavirin Doses

| Patient | | | Regimen | Total Number of |
|---|---|---|---|---|
| Weight* | | Total Daily Dose | (200 mg/each) | Capsules Dose |
| <65 | kg | 800 mg | 400 mg/400 mg | 2 capsules am/ |
| | | | divided doses BID | 2 capsules pm |
| 65-85 | kg | 1000 mg | 400 mg/600 mg | 2 capsules am/ |
| | | | divided doses BID | 3 capsules pm |
| 86-105 | kg | 1200 mg | 600 mg/600 mg | 3 capsules am/ |
| | | | divided doses BID | 3 capsules pm |
| >105 | kg | 1400 mg | 600 mg/600 mg | 4 capsules am/ |
| | | | divided doses BID | 3 capsules pm |

Ribavirin will be administered by the oral route twice daily (BID) at doses ranging from 8000 to 4200 mg per day. Ribavirin doses are defined in the following Table A:
*Conversion of lbs. to kg can be done by dividing by 2.2

All ribavirin doses will be administered on a BID schedule. If an adverse event occurs for which a dose reduction is required, the Total Daily Dose should be adjusted as described in, Table B.

TABLE B

| | | Number of capsules |
|---|---|---|
| Protocol Dose | Dose reduction | (200 mg) |
| PEG-interferon alfa-2b | PEG-interferon alfa-2b | |
| 1.5 mcg/kg QW | 1.0 mcg/kg QW | |
| Ribavirin 1200 mg | Ribavirin 1000 mg daily | 3 caps AM/2 caps PM |
| daily | 2^{nd} dose reduction | 2 caps AM/2 caps PM |
| | allowed to 800 mg daily | |
| Ribavirin 1000 mg | Ribavirin 800 mg daily | 2 cap AM/2 caps PM |
| | 2^{nd} dose reduction | 2 caps AM/1 cap PM |
| | allowed to 600 mg/daily | |

Supplies of PEG-Intron(pegylatedinterferon alfa-2b)

Commercially available PEG-Intron which is commercially available from Schering Corporation, Kenilworth, N.J. will be used.

Administration of PEG-Intron(pegylatedinterferon alfa-2b)

PEG-Intron will be administered by the subcutaneous route. During the first 48 hours of therapy, the study physician, physician coordinator or nursing staff should be easily accessible to the patient since adverse events with Intron A are typically the most severe following the first injection. Flu-like symptoms, fever, chills, fatigue, and malaise occur in most patients within two to eight hours after the initial dose of Intron A. Initial reactions are generally mild to moderate in nature, and in most patients tachyphylaxis of these symptoms occurs after 3-5 doses. Patients may be premedicated with standard doses of acetaminophen or non-steroidal anti-inflammatory drugs one hour prior to administration of the study medication. Please see the PEG-Intron Package Insert for additional information.

Antioxidant Therapy Supplies and Administration

Vitamin E Supplies and Administration

Vitamin E will be supplied in capsule form of 400 IU strength. The capsules will be supplied by Schering Corporation and dispensed by study staff through week 24 of the antiviral combination therapy. Patients will take these capsules on a daily BID basis

Vitamin C Supplies and Administration

Vitamin C will be supplied in the form 500 mg_tablets. The tablets will supplied by Schering Corporation and dispensed by study staff through week 24 of the antiviral combination therapy

S-Adenosyl-L-Methionine Supplies and Administration

S-Adenosyl-L-Methionine will be supplied in the form of tablets. The tablets will be supplied by Schering Corporation and dispensed by study staff through week 24 of the antiviral combination therapy

Storage of Study Medications

All study medications may be stored at room temperature

### Duration of Treatment

Duration of treatment with the PEG-Intron and ribavirin combination therapy will be for up to 48 weeks. Duration of treatment with the antioxidant regimen will be for up to 28 weeks. Antioxidant therapy after week 24 of the antiviral combination therapy will be at the discretion of the investigator.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed:

1. A method for treating a patient having a susceptible viral infection which comprises administering to said patient a therapeutically effective amount of ribavirin for a time sufficient to lower viral-RNA in association with a therapeutically effective amount of an antioxidant therapy comprising S-adenosyl methionine for a time sufficient to ameliorate ribavirin-related hemolysis.

2. The method of claim 1 wherein the antioxidant therapy further comprises at least one of Vitamin A, Vitamin E, Vitamin C, coenzyme-Q10, BHA, BHT, 2-oxo-4-thiazolidinecarboxylic acid, N-acetylcysteine, selenium, panavir, silybum marianum, lycopene, or a pharmaceutically acceptable salt or ester thereof.

3. A method of treating a patient having chronic HCV infection which comprises administering to said patient a therapeutically effective amount of a combination therapy of interferon-alfa and ribavirin for a time sufficient to lower HCV-RNA in association with a therapeutically effective amount of an antioxidant therapy comprising S-adenosyl methionine for a time sufficient to ameliorate ribavirin-related hemolysis.

4. The method of claim 3 wherein the interferon alfa is interferon alfa-2a, interferon-alfa-2b, pegylated interferon alfa-2a, pegylated interferon alfa-2b, or a consensus interferon or a purified interferon alfa product.

5. The method of claim 3 wherein the antioxidant therapy futher comprises at least one Vitamin A, Vitamin E, Vitamin C, coenzyme-Q10, BHA, BHT, 2-oxo-4-thiazolidinecarboxylic acid, N-acetylcysteine, selenium, panavir, silybum marianum, lycopene, or a pharmaceutically acceptable salt or ester thereof.

6. The method of claim 3 wherein the antioxidant therapy comprises Vitamin C, Vitamin E, and S-adenosyl methionine or a pharmaceutically acceptable salt or ester thereof.

7. The method of claim 6 wherein S-adenosyl L-methionine is used.

8. The method of claim 3 wherein the combination therapy comprises 3MIU TIW of interferon alfa-2b and about 600 mg to about 2000 mg/day of ribavirin and the antioxidant therapy are administered.

9. The method of claim 3 wherein the combination therapy and the antioxidant therapy are administered for a time period of at least about 24 weeks.

10. The method of claim 3 wherein the combination therapy and the antioxidant therapy are administered for a time period at least about 48 weeks.

11. The method of claim 3 wherein the combination therapy and the antioxidant therapy are administered for a time period of at least about 72 weeks

12. The method of claim 3 wherein the antioxidant therapy is administered for at least four weeks prior to administering the combination therapy.

13. The method of claim 3 wherein the combination therapy comprises about 0.5 to about 3.0 μg/kg/week of pegylated interferon alfa-2b and about 600 to about 2000 mg/day of ribavirin.

14. The method of claim 3 wherein the combination therapy comprises about 0.5 to about 3.0 μg/kg/week of pegylated interferon alfa-2b and at least about 13 mg/kg/day of ribavirin.

15. A method of treating a patient having a chronic HCV infection which comprises administering to said patient a therapeutically effective amount of a combination therapy of pegylated interferon alfa and ribavirin for a time period sufficient to lower detectable HCV-RNA in association with a therapeutically effective amount of an antioxidant therapy sufficient to ameliorate ribavirin-related hemolysis.

16. The method of claim 15 wherein the antioxidant therapy comprises is at least one of Vitamin A, Vitamin E, Vitamin E, Vitamin C, coenzyme-Q10, BHA, BHT, 2-oxo-4-thiazolidinecarboxylic acid, N-acetyl cysteine, selenium, panavir, silybum marianum, lycopene, S-adenosyl methionine or a pharmaceutically acceptable salt or ester thereof.

17. The method of claim 15 wherein the antioxidant therapy comprises Vitamin C, Vitamin E, and S-adenosyl methionine.

18. The method of claim 17 wherein S-adenosylL-methionine is used.

19. The method of claim 15 wherein the pegylated interferon alfa is pegylated interferon alfa-2a, pegylated interferon alfa-2b, a pegylated consensus interferon or a pegylated purified interferon alfa product.

20. The method of claim 15 wherein the combination therapy and the antioxidant therapy are administered for a time period at least about 24 weeks

21. The method of claim 15 wherein the combination therapy and the antioxidant therapy are administered for a time period at least about 48 weeks.

22. The method of claim 15 wherein the combination therapy and the antioxidant therapy are administered for a time period at least about 72 weeks.

23. The method of claim 15 wherein the combination therapy comprises about 0.5 to about 3.0 μg/kg/week of pegylated interferon alfa-2b and about 600 to about 2000 mg/day of ribavirin.

24. The method of claim 15 wherein the combination therapy comprises about 0.5 to about 3.0 μg/kg/week of pegylated interferon alfa-2b and at least about 13 mg/kg/day of ribavirin.

25. The method of claim 15 wherein the antioxidant therapy is administered for at least four weeks prior to administering the combination therapy.

26. A method of treating a patient having a chronic HCV infection which comprises administering to said patient a combination therapy of about 0.5 to about 3.0 μg/kg/week of pegylated interferon alfa-2b and about 600 to about 2000 mg/day of ribavirin in association with a therapeutically effective amount of an antioxidant therapy sufficient to ameliorate ribavirin-related hemolysis the antioxidant therapy for a time period of at least about 24 weeks

27. The method of claim 26 wherein the antioxidant therapy is administered for at least four weeks prior to administering the combination therapy.

28. The method of claim 26 wherein the combination therapy and the antioxidant therapy are administered for a time period of at least about 48 weeks

29. The method of claim 26 wherein the combination therapy and the antioxidant therapy are administered for a time period of at least about 72 weeks

30. A method of treating a patient having a chronic HCV infection which comprises administering to said patient a combination therapy of about 0.5 to about 3.0 μg/kg/week of pegylated interferon alfa-2b and about 800 to about 2000 mg/day of ribavirin for a time period sufficient to lower detectable HCV-RNA in association with a therapeutically effective amount of an antioxidant therapy sufficient to ameliorate ribavirin-related hemolysis the antioxidant therapy.

31. The method of claim 30 wherein the antioxidant therapy is administered for at least four weeks prior to administering the combination therapy.

32. The method of claim 30 wherein the combination therapy and the antioxidant therapy are administered for a time period of at least about 24 weeks

33. The method of claim 30 wherein the combination therapy and the antioxidant therapy are administered for a time period of at least about 48 weeks

34. The method of claim 30 wherein the combination therapy and the antioxidant therapy are administered for a time period of at least about 72 weeks.

35 A method of treating a patient having a chronic HCV infection which comprises administering to said patient a combination therapy of about 0.5 to about 3.0 μg/kg/week of pegylated interferon alfa-2b and at least 13 mg/kg/day of ribavirin in association with a therapeutically effective amount of an antioxidant therapy sufficient to ameliorate ribavirin-related hemolysis the antioxidant therapy.

36. The method of claim 35 wherein the antioxidant therapy is administered for at least four weeks prior to administering the combination therapy.

37. The method of claim 35 wherein the combination therapy and the antioxidant therapy are administered for a time period of at least about 24 weeks

38 The method of claim 35 wherein the combination therapy and the antioxidant therapy are administered for a time period of at least about 48 weeks

39. The method of claim 35 wherein the combination therapy and the antioxidant therapy are administered for a time period of at least about 72 weeks
